(19) United States
(12) Patent Application Publication
(10) Pub. No.: US 2010/0093641 A1
(43) Pub. Date: Apr. 15, 2010

(54) SILK PROTEIN-MIMICKING PEPTIDES AND COMPOSITIONS FOR PREVENTING OR TREATING CRANIAL NEUROPATHIES COMPRISING THE SAME

(75) Inventors: Sung Su Kim, Gyeonggi-do (KR); Do Yeon Lee, Seoul (KR); Yoo Hun Noh, Seoul (KR)

Correspondence Address: BUCHANAN, INGERSOLL & ROONEY PC, POST OFFICE BOX 1404, ALEXANDRIA, VA, 22313-1404, US

(73) Assignee: BRAINGUARD CO.,LTD., SEOUL (KR)

(21) Appl. No.: 12/445,284

(22) PCT Filed: Oct. 17, 2007
(86) PCT No.: PCT/KR07/05084

§ 371 Date: Apr. 10, 2009

(30) Foreign Application Priority Data
Oct. 7, 2006 (KR) 10-2006-0100643

(51) Int. Cl. A61K 38/08; A61K 38/07; A61K 38/03; C07K 7/00; C07K 5/00; C07K 4/00; A61P 25/00; A61P 25/18; A61P 25/16; A61P 25/24

(52) U.S. Cl. 514/16; 514/17; 514/18; 530/328; 530/329; 530/330; 530/331

(57) ABSTRACT

The present invention relates to a silk protein-mimicking peptide represented by the following Formula 1, a composition for preventing or treating a brain disease, a composition for improving a brain or cognitive function and a composition for preventing or treating diseases, disorders or conditions associated with oxidative stress comprising the peptide:

Gly-Xₐₐ₁-Gly-Xₐₐ₂ (1)
wherein Xₐₐ₁ is Ala, Val, Ser, Tyr, Asp, Glu, Thr, Met, Ile, Leu, Phe, His, Lys or Arg; Xₐₐ₂ is Ala, Tyr, Val, Ser, Asp, Glu, Thr, Met, Ile, Leu, Phe, His, Lys or Arg; or Xₐₐ₂ represents Ala, Tyr, Val, Ser, Asp, Glu, Thr, Met, Ile, Leu, Phe, His, Lys or Arg residue linked to Gly-Xₐₐ₃; and Xₐₐ₃ is Tyr, Val, Ala, Ser, Asp, Glu, Thr, Met, Ile, Leu, Phe, His, Lys or Arg.

### BACKGROUND OF THE INVENTION

1. Field of the Invention

The present invention relates to silk protein-mimicking peptides having neuroprotective activities and brain function-enhancing activities and their uses.

2. Description of the Related Art

Cerebral apoplexy (or stroke) refers to a cerebral disease, a most highly ranked cause of death in Korea, caused by rupture or obstruction of blood vessels in brain and results in abnormalities in some cerebral tissues. The death rate due to the above disease has been on the rise because of extended life span due to industrialization and development of medical science. Stroke may develop in any part of a body and cause dysfunction of the part accordingly. Medically, stoke is divided into ‘ischemic stroke’ and ‘hemorrhagic stroke’, and the former, which is more closely related to hypertension and arteriosclerosis, shows a relatively higher rate of recurrence. Ischemic stroke is caused by obstruction in from any blood vessel around a neck (e.g. a carotid artery) to any in brain. As a result, cerebral infarction occurs and the function of that region may not be recovered for good. Therefore, the most important thing in treating stroke is prevention of cerebral ischemia itself together with prevention of risk factors such as hypertension, diabetes and hypercholesterolemia. According to current therapies, where stroke is developed to infarction, the prevention of secondary brain damages by alleviation of cerebral edema and promotion of circulation at ischemic regions becomes the utmost efforts.

Examples of substances currently used for neuronal protection are excitatory amino acid antagonists such as ganglioside and nimodipine, and GABA agonists such as clomethiazole. Magnesium sulfate and glycine antagonist are under Phase II clinical trial and a large-scale clinical trial is being performed about piracetam. However, the conventional neuroprotective agents are mainly aimed at acting on different steps in ischemia development, and thus there still remains a need to develop a composite agent acting simultaneously on several steps with little side effects and drug complications.

Furthermore, since ischemic stroke is abruptly developed without specific prognosis, a functional food, which may constantly prevent ischemia and inhibit post-ischemic neuronal apoptosis, has been considered to be more effective than drugs to prevent ischemic stoke itself.

U.S. Pat. No. 6,245,757 discloses a use of progestin for treating cell impairment by ischemia. U.S. Pat. No. 6,380,193 discloses a pharmaceutical composition comprising poly(adenosine 5′-diphospho-ribose) polymerase inhibitor for treating stroke. Also, U.S. Pat. No. 6,288,041 discloses a pharmaceutical composition comprising sialic acid derivatives for treating stroke.

Parkinson's disease (PD) is one of neuronal degenerative diseases that may cause impairment in movement and intelligence and was first reported by James Parkinson in 1817. In US, the attack rate of this disease is about 100-150 persons per 100,000 people. The number of current patients is about 750,000-1,000,000 and about 60,000 new patients are added to the list each year. Considering the global trend of aging society, its incidence rate is also expected to increase in Korea. Pathologically, PD induces loss of dopamine neuronal cells in substantia nigra and decrease of dopamine in caudate nucleus and putamen, followed by impairments in movement and intelligence such as tremor, bradykinesia, rigidity and disturbance of posture.

Drugs that can supplement functions of dopamine in brain, or prevent or delay destruction of neuronal cells, or control the accompanying symptom such as depression have been used to treat Parkinson's disease. Examples of those drugs are madopar (levodopa, L-dopa; dopamine precursor), bromidine (dopamine receptor agonist), lisuride, artane (anti-acetylcholine) and cogentin. Of these drugs, levodopa is known to be the most effective in treating Parkinson's disease by supplementing dopamine level in brain. However, when administered for more than 3-5 years, the levodopa shows side effects such as a shortened effective time (wearing-off) or large fluctuation in motion controlling function (on-off phenomenon) and abnormal motion symptom (diskinesia) (Freed et. al., N. Engl. J. Med. 327:1549-55(1992)).

Further, surgical treatments for Parkinson's disease have been also used, and their examples include thalamotomy, pallidotomy, deep brain stimulation and neuronal cell transplantation. However, a lasting time of efficacy differs significantly from patient to patient along with serious side effects such as hypophonia accompanying operation, dysarthria and a decline in memory (Ondo et. al., Neurology 50:266-270 (1998); Shannon et. al., Neurology 50:434-438(1998)).

Treatment of Alzheimer's disease has been recently focused on the fact that Alzheimer's disease may be caused by impaired cholinergic signaling and transmission in cerebral cortex and hippocampus (Bartus et al., Science. 217(4558): 408-14(1982)); Coyle et al., Science. 219(4589):1184-90(1983)). Because this region in brain is associated with memory and intelligence, functional defect in this region may cause loss of memory and intelligence. Although the process of impairment in neuronal signaling is still controversial, senile plaque and neurofibrillary tangle (NFT) are considered as main causes. Senile plaque due to the accumulation of amyloid beta (Aβ) is a notable feature of this disease, and Alzheimer disease may be confirmed by a postmortem examination (Khachaturian, Arch. Neurol. 42(11):1097 105(1985)).

As a way of treating Alzheimer's disease, methods of increasing or maintaining acetylcholine level to inhibit the impairment of cholinergic signaling or causing acetylcholine to acts more effectively on transmission of neuronal cells have been provided. Therefore, patients of Alzheimer's disease are administered with compounds for increasing activity of acetylcholine. The most effective way is to rapidly decompose acetylcholine in synapse, thus inhibiting activity of acetylcholinesterase, and these inhibitors (e.g. tacrine, donepezil and rivastigmine) have been approved by FDA and currently on market. Despite their effectiveness in preventing further destructive progress of the disease, they are not applied to recover patients to pre-illness level.

Some compounds are aimed to improve neuronal condition and maintain aged cells in good function. For example, NGF or estrogen acts as neuroprotecting agents to delay neurodegeneration and anti-oxidants decreases cell damage caused by oxidation of cells. Alzheimer's disease becomes serious as amyloid beta peptide is accumulated in neuritic space, and amyloid precursor protein (APP) is considered to play a role in combination with proteinase in cells such as α-, β-and γ-secretases. However, since the mechanisms of amyloid beta formation are not still elucidated, it is not impossible to control the formation of the amyloid beta protein.

It is not certain how the accumulation of amyloid beta acts on neuronal signaling. Abnormally cleaved APP induces generation of amyloid beta, and plaques are induced by the accumulation of the amyloid beta protein. Thus, various factors involved in the cleavage reaction (e.g. inflammation) increase phosphorylation of the tau protein and increase the accumulation of paired helical filament (PHF) in combination with NET, resulting in neurodegeneration and finally expedition of dementia of Alzheimer's type.

Even though a multitude of researchers have suggested therapies to treat Alzheimer's disease, the treatment of Alzheimer's disease is just focused on temporal alleviation of the symptom instead of restoring disease process. Biological information on Alzheimer's disease becomes increasing, but successful clinical results are not yet published.

U.S. Pat. No. 5,532,219 discloses a pharmaceutical composition comprising 4,4′-diaminodiphenylsulfone for treating Alzheimer's disease. U.S. Pat. No. 5,506,097 discloses a pharmaceutical composition comprising para-amidinophenylmethanesulfonyl fluoride or Ebelactone A for treating Alzheimer's disease. U.S. Pat. No. 6,136,861 discloses a pharmaceutical composition comprising bicyclo[2.2.1]heptane.

Meanwhile, stress is becoming major problem in health in modern society, and it is reported that, in Korea, ⅓ of the twenties usually experience much stress, and that the women suffer from stress than men in their teens. Strength of stress depends on personality, interest, means of relief from stress, surrounding environment, controlling ability of a person, and stress is usually followed by depression. Depression may results in suicide, and is considered to be a very important disorder because of its high rate of occurrence and recurrence. Depression has been reported to be caused by impairment of neurotransmitters such as adrenaline, dopamine or serotonin, and followed by cerebral impairment. Although tricylic antidepressant (TCA), it has drawbacks of having serious side effects. Especially, amitriptyline is a well-known therapeutics but it has been reported to have various side effects. Fluoxetine, a selective serotonin re-uptake inhibitor (SSRI) developed in the US in 1980s, ranked 7 among 20 international drugs because it overcame the problems of TCA and increase the drug compliance. However, SSRI showed little improvement in efficacy compared with TCA and still has serious drug interference.

Moreover, neuronal disturbance is continuously induced by stress and there is no way to inhibit the recurrence after medicinal treatment of depression, and thus a long-term administration with a lowered dose is only used at present. Therefore, it is very important to develop substances with superior activity of inhibiting neuronal apoptosis and correcting neuronal transmission system. Furthermore, functional foods with anti-depression activity are also important to be developed, as considering a tendency of avoiding visiting treatment institution and overlooking the induction of disturbance in serotonin neuronal system and cerebral impairment.

As representative examples of effort to develop medicine for preventing and treating neuronal degenerative disease, U.S. Pat. No. 6,020,127 discloses genes encoding proteins suppressing neuronal apoptosis from human chromosome 5q13, and U.S. Pat. No. 6,288,089 disclose pyridyl imidazole derivatives for treating neuronal degenerative disease by suppressing apoptosis of dopaminergic neuron.

However, there are little known about substances having significant neuroprotective activities on various cerebral diseases as well as enhancement activities in brain functions. In addition, synthetic peptides having accurately identified sequences have not been yet suggested for neuroprotection.

Throughout this application, several patents and publications are referenced and citations are provided in parentheses. The disclosure of these patents and publications is incorporated into this application in order to more fully describe this invention and the state of the art to which this invention pertains.

### DETAILED DESCRIPTION OF THIS INVETNION

The present inventors have made intensive researches to develop synthetic peptides exhibiting activities of natural-occurring-derived silk proteins or silk peptides by designing novel sequences of peptides and analyzing their activities. As a result, we have discovered that peptides represented by the Formula 1 possess activities similar to those of natural-occurring silk proteins or silk peptides, eventually accomplishing the present invention.

Accordingly, it is an object of this invention to provide a silk protein-mimicking peptide.

It is another object of this invention to provide a composition for preventing or treating a brain disease.

It is still another object of this invention to provide a composition for improving a brain or cognitive function.

It is further object of this invention to provide a composition for preventing or treating diseases, disorders or conditions associated with oxidative stress.

It is still further object of this invention to provide a method for preventing or treating a brain disease.

It is another object of this invention to provide a method for improving a brain or cognitive function.

It is still another object of this invention to provide a method for preventing or treating diseases, disorders or conditions associated with oxidative stress.

Other objects and advantages of the present invention will become apparent from the following detailed description together with the appended claims and drawings.

In one aspect of this invention, there is provided a silk protein-mimicking peptide represented by the following Formula 1:

Gly-Xₐₐ₁-Gly-Xₐₐ₂ (1)

wherein Xₐₐ₁ is Ala, Val, Ser, Tyr, Asp, Glu, Thr, Met, Ile, Leu, Phe, His, Lys or Arg; Xₐₐ₂ is Ala, Tyr, Val, Ser, Asp, Glu, Thr, Met, Ile, Leu, Phe, His, Lys or Arg; or Xₐₐ₂ represents Ala, Tyr, Val, Ser, Asp, Glu, Thr, Met, Ile, Leu, Phe, His, Lys or Arg residue linked to Gly-Xₐₐ₃; and Xₐₐ₃ is Tyr, Val, Ala, Ser, Asp, Glu, Thr, Met, Ile, Leu, Phe, His, Lys or Arg.

The present inventors have made intensive researches to develop synthetic peptides exhibiting activities of natural-occurring-derived silk proteins or silk peptides by designing novel sequences of peptides and analyzing their activities. As a result, we have discovered that peptides represented by the Formula 1 possess activities similar to those of natural-occurring silk proteins or silk peptides.

The present invention provides silk protein-mimicking peptides having novel amino acid sequences.

The term used herein “silk protein-mimicking peptide” refers to synthetic peptides having biological activities of natural-occurring silk proteins or silk peptides. The abbreviation of silk protein-mimicking peptide is SMP (Silk Mimicking Peptide).

The term used herein “peptide” refers to a linear molecule formed by linking amino acid residues through peptide bonds. The length of the present peptides is typically 4-50, preferably 4-40, more preferably 4-30, and most preferably 4-20 amino acid residues.

According to a preferred embodiment, Xₐₐ₁ is Ala, Val, Ser or Tyr, more preferably, Ala or Val.

According to a preferred embodiment, Xₐₐ₂ is Ala, Tyr, Val or Ser, more preferably, Ala or Tyr, most preferably Ala.

According to a preferred embodiment, Xₐₐ₂ is Ala, Tyr, Val or Ser residue linked to _{Gly-X}ₐₐ₃; and Xₐₐ₃ is Tyr, Val, Ala or Ser. In such a peptide, the Formula 1 is specifically described by the following Formula 2:

Gly-Xₐₐ₁-Gly-Xₐₐ₂-Gly-Xₐₐ₃ (2)

wherein, Xₐₐ₃ is Tyr, Val, Ala or Ser.

More preferably, in the Formula 2, Xₐₐ₃ is Tyr or Val.

Alternatively, Xₐₐ₃ further comprises Gly-Xₐₐ₄; and Xₐₐ₄ is Tyr, Ala, Val, Ser, Asp, Glu, Thr, Met, Ile, Leu, Phe, His, Lys or Arg.

Preferably, Xₐₐ₅ is Tyr, Ala or Val.

Most preferably, the peptides of the present invention comprise the amino acid sequence selected from the group consisting of SEQ ID NOs. 1-4.

The peptides of the invention may be prepared by conventional chemical synthesis processes known to one of skill in the art, in particular, solid-phase synthesis techniques (Merrifield, J. Amer. Chem. Soc. 85:2149-54(1963); Stewart, et al., Solid Phase Peptide Synthesis, 2nd. ed., Pierce Chem. Co.: Rockford, 111(1984)).

Even though the peptides of this invention per se have higher stability, their modification with protection groups enables to have much higher stability. Exemplary protection group includes amino acids, acetyl group, fluorenyl methoxy carbonyl group, formyl group, palmitoyl group, myristyl group, stearyl group or polyethylene glycol (PEG). Most preferably, the acetyl protection group is linked to the peptides of this invention.

Although the protection group may be linked to various positions of the present peptides, it is preferable that it is linked to N-or C-terminal of the present peptides.

The most striking feature of the present peptides in view of their sequences is that a Gly residue is regularly positioned once in two consecutive amino acid residues. Therefore, it could be understood to one of skill in the art that variations or modifications comprising amino acid sequences represented by the Formula 1 and the Gly residue positioning pattern are encompassed by the present invention, irrespective of their length. In other words, the Formula 1 is described not to limit the length of the present peptides but to clearly define the present peptides.

The silk protein-mimicking peptide possess various biological and physiological activities of natural-occurring silk proteins and peptides (hydrolysates of silk proteins, see Korean Pat. No. 0494357), including protection of neuronal cells, treatment of brain diseases or disorders, improvement of brain or cognitive functions, inhibition of oxidative stress and improvement of skin moisture, as well as higher stability. In particular, the peptides of this invention are very effective in protection of neuronal cells, treatment of brain diseases or disorders, improvement of brain or cognitive functions and inhibition of oxidative stress.

In addition, it would be noteworthy that the peptides of this invention may be easily delivered to in vivo targets because they have much lower molecular weights and higher stability than natural-occurring silk proteins and peptides. The peptides of this invention SMPs themselves or with help of other delivery systems show significant in vivo delivery potential. In this context, the peptides of this invention are advantageous in development of drugs and functional foods.

Conventional silk proteins and silk peptides are not homogenous, making them restrictive. For instance, drugs or functional food with constant quality may not be manufactured using conventional silk proteins and silk peptides.

The SMPs of this invention having activities of conventional silk proteins or silk peptides could overcome the shortcomings described above.

In another aspect of this invention, there is provided a composition for preventing or treating a brain disease, comprising an effective amount of the silk protein-mimicking peptide described above.

In still aspect of this invention, there is provided a method for preventing or treating a brain disease, comprising administering to a subject a composition comprising an effective amount of the silk protein-mimicking peptide described above.

In further aspect of this invention, there is provided a composition for improving a brain or cognitive function, comprising an effective amount of the silk protein-mimicking peptide described above.

In still further aspect of this invention, there is provided a method for improving a brain or cognitive function, comprising administering to a subject a composition comprising an effective amount of the silk protein-mimicking peptide described above.

In another aspect of this invention, there is provided a composition for preventing or treating diseases, disorders or conditions associated with oxidative stress, comprising an effective amount of the silk protein-mimicking peptide described above.

In still another aspect of this invention, there is provided a method for preventing or treating diseases, disorders or conditions associated with oxidative stress, comprising administering to a subject a composition comprising an effective amount of the silk protein-mimicking peptide described above.

The present composition may be applied to various brain diseases, preferably, neurodegenerative diseases, ischemia-reperfusion injury and mental disorders. The neurodegenerative disease treated by the present composition includes preferably dementia, Huntington's disease, Parkinson's disease and amyotrophic lateral sclerosis. The ischemia or reperfusion injury treated by the present composition includes preferably ischemic stroke. The mental disorder treated by the present composition includes preferably depression, schizophrenia and post traumatic stress disorder.

The present composition is very effective in improving a brain or cognitive function. Preferably, the brain or cognitive function is a learning ability, a memory ability or a concentration ability. In addition, the present composition is very effective in ameliorating the aggravation of brain or cognitive functions associated with brain diseases.

The efficacies of the present composition relating to nervous system are ascribed mostly to their neuroprotective activity. As used herein, the term “neuronal cell” includes neuron, neuronal supporting cell, Glia and Schwann cell constituting central nervous system, brain, brainstem, spinal cord or a connecting structure between central nervous system and peripheral nervous system. As used herein, the term “neuroprotective activity” refers to the effects of reducing or ameliorating nervous insult, and protecting or reviving neuronal cells that has suffered nervous insult. As used herein, the term “nervous insult” refers to any damage to neuronal cell or tissue resulting from various causes such as metabolic, toxic, neurotoxic and chemical causes.

The neuroprotective activity of the present peptides is exhibited via various mechanisms such as inhibition of neuronal cell death including necrosis and apoptosis of neuronal cell. The inhibition of neuronal apoptosis may be accomplished by inhibiting the generation of reactive oxygen species or protecting mitochondrial functions (see Examples).

In addition to this, the present composition is very effective in treating diseases, disorders or conditions associated with oxidative stress.

The oxidative stress induced by substances with high oxidizing potential e.g., reactive oxygen species (e.g., superoxide and peroxide) is a main cause of various diseases. The oxidative stress has been reported as aging-inducing agents. The peptides of this invention decrease the oxidative stress by inhibiting the generation of reactive oxygen species.

According to a preferred embodiment, the disease, disorder or condition associated with oxidative stress is aging; central nerve system disorders including trauma, cerebral palsy and diabetic neuropathy; cardiovascular diseases including intermittent claudication and arteriosclerosis; cataract; musculoskeletal diseases including arthritis; or disorders associated with environments causing the generation of reactive oxygen species including ionizing radiation-associated disorder, cancer chemotherapy-associated disorder and carcinogen expose-associated disorder.

According to a preferred embodiment, the peptides of this invention protects brain from traumatic damages by inhibiting the generation of interleukin-1β (IL-1β) or tumor necrosis factor-α (TNF-α).

The present composition may be formulated into a pharmaceutical composition and food composition.

For formulating pharmaceutical compositions, the present composition comprises (i) a pharmaceutically effective amount of the silk protein-mimicking peptide; and (ii) a pharmaceutically acceptable carrier. The term used herein “pharmaceutically effective amount” refers to an amount suitable to show and accomplish efficacies and activities of the peptide of this invention.

In the pharmaceutical compositions of this invention, the pharmaceutically acceptable carrier may be conventional one for formulation, including carbohydrates (e.g. lactose, amylase, dextrose, sucrose, sorbitol, mannitol, starch and cellulose), acacia rubber, calcium phosphate, alginate, gelatine, calcium silicate, fine crystallite cellulose, polyvinylpyrrolidine, cellulose, water, syrup, salt solution, alcohol, Arabian rubber, vegetable oil (e.g. corn oil, cotton seed oil, soybean oil, olive oil and coconut oil), poly(ethylene glycol), methyl cellulose, methylLydroxy benzoate, propylLydroxy benzoate, talc, magnesium stearate and mineral oil. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

A pharmaceutical composition of this invention may be administered orally or parenterally. For non-oral administration, intravenous injection, subcutaneous injection or intramuscular injection may be employed.

A suitable dose of the pharmaceutical composition of the present invention may vary depending on pharmaceutical formulation methods, administration methods, the patient's age, body weight, sex, severity of diseases, diet, administration time, administration route, an excretion rate and sensitivity for a used pharmaceutical composition. Physicians of ordinary skill in the art can determine an effective amount of the pharmaceutical composition for desired treatment. Preferably, the pharmaceutical composition of the present invention is administered with a daily dose of 0.001-100 mg/kg (body weight). The administration may be done once or several times per day.

According to the conventional techniques known to those skilled in the art, the pharmaceutical composition may be formulated with pharmaceutically acceptable carrier and/or vehicle as described above, finally providing several forms including a unit dose form and a multi-dose form. Non-limiting examples of the formulations include, but not limited to, a solution, a suspension or an emulsion in oil or aqueous medium, an extract, an elixir, a powder, a granule, a tablet and a capsule, and may further comprise a dispersion agent or a stabilizer.

The present composition may be prepared to provide a food composition, in particular a health food composition. The food composition may comprise conventional additives for preparing food compositions, e.g., proteins, carbohydrates, lipids, nutritive substances and flavors. For example, where the food composition of this invention is provided as a drink, it may further comprise flavors and natural carbohydrates as well as peptides as active ingredients. Non-limiting examples of natural carbohydrates include, but not limited to, monosaccharide (e.g,, glucose and fructose), disaccharide (e,g., maltose and sucrose), oligosaccharide, polysaccharide (e.g., dextrin and cyclodextrin) and sugar alcohol (e.g., xylitol, sorbitol and erythritol). Non-limiting examples of flavors include, but not limited to, natural flavors (e.g., thaumatin and extract of Stevia) and synthetic flavors (e.g., saccharin and aspartame).

The features and advantages of this invention are summarized as follows:

(i) The silk protein-mimicking peptides of this invention have biological and physiological activities of natural-occurring silk proteins or silk peptides, as well as excellent stability.

(ii) Since the peptides of this invention have much lower molecular weight, their application becomes wider than conventional silk proteins or silk peptides having higher molecular weights.

(iii) Since the peptides of this invention have much lower molecular weight, their in vivo delivery and bioavailability are considerable.

(iv) Using the peptides of this invention, homogeneous drugs or foods may be provided. Therefore, the peptides of this invention are very advantageous in manufacturing drugs and food with constant quality.

(v) In particular, the peptides of this invention exhibit plausible neuroprotective activities to prevent and treat various brain diseases.

As described hereinabove, te silk protein-mimicking peptides of this invention have biological and physiological activities of natural-occurring silk proteins or silk peptides, as well as excellent stability. Since the peptides of this invention have much lower molecular weight, their application becomes wider than conventional silk proteins or silk peptides having higher molecular weights. Since the peptides of this invention have much lower molecular weight, their in vivo delivery and bioavailability are considerable. Using the peptides of this invention, homogeneous drugs or foods may be provided. Therefore, the peptides of this invention are very advantageous in manufacturing drugs and food with constant quality. In particular, the peptides of this invention exhibit plausible neuroprotective activities to prevent and treat various brain diseases.

### BRIEF DESCRIPTION OF THE DRAWINGS

FIG. 1 is a photograph showing neuroprotective activities of the present peptides in dopaminergic neuronal cells treated by 6-OHDA (6-hydoxydopamine). The control represents a group treated with only 6-OHDA.

FIG. 2 represents a graph showing prevention effects of the present peptides on behavior disorders in Parkinson's animal models with apoptotic dopminergic neuronal cells by 6-OF-IDA.

FIG. 3 represents neuroprotective activities of the present peptides on amyloid protein-induced neuronal death in hippocampus.

FIG. 4 represents neuroprotective effects on neurotoxicity of the amyloid beta protein. The experiment was performed according to the passive avoidance test.

FIGS. 5a-5b represent neuroprotective effects on neurotoxicity of the amyloid beta protein. The experiment was performed according to the water maze test.

FIGS. 6a-6b represent neuroprotective effects on neurotoxicity of the amyloid beta protein. FIG. 6a is the results of nuclear fragmentation analysis and FIG. 6b is the results of cell viability. In Figures, oligo 1 and oligo 2 represent SMP-1 and SMP-4, respectively.

FIGS. 7a-7c represent neuroprotective effects on neuronal cell death by reactive oxygen species.

FIGS. 8a-8c represent neuroprotective effects on neuronal cell death by impairment of mitochondrial functions.

FIGS. 9a-9b represent neuroprotective effects on neuronal cell damages caused by high glucose level.

FIGS. 10a-10b represent neuroprotective effects on traumatic neuronal damages imposed directly on brain/

FIG. 11 represents the influence of the present peptides on levels of pro-inflammatory cytokines, interleukin-β and TNF-α.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Preparative Example 1

### Preparation of Peptides

We designed silk peptide-mimicking peptides having various amino acid sequences and showing physiological activities of silk proteins. The designed peptides was chemically synthesized in Peptron, Inc.(Korea). The representative examples of synthesized peptides are summarized in Table 1, called as “SMP (Silk Mimicking Peptide).

TABLE 1

| Peptides | Sequence |
|---|---|
| SMP-1 | GAGAGVGY |
| SMP-2 | GVGY |
| SMP-3 | GAGAGY |
| SMP-4 | GVGAGY |

### Example 1

### Analysis of Neuroprotective Effect on Dopaminergic Neuronal Cell Death induced by 6-OHDA

To analyze neuroprotective effects of the present peptides SMPs, their influence on the death of dopaminergic neurons, a main cause of Parkinson's disease, was tested. 6-OHDA (6-hydroxydopamine) was injected into brain tissues using a stereotaxic system and the death of dopaminergic neurons was observed by histochemcial staining.

5-week old male mice (SAMTAKO, Korea) were used. Four-week old mice were purchased and adapted for one week, followed by inducing the death of neuronal cells using a stereotaxic system (KOFT, CA, USA). In these experiments, 6-OHDA (3 μg/μl in normal saline) was injected into the desired region (0.0 mm posterior to bregma; 2.0 mm lateral to midline; 3.0 mm ventral to the dura) by use of the stereotaxic system, resulting in establishment of animal models. 6-OHDA was injected into substantial nigra in which neurites of dopminergic neurons are connected and sustained death of dopminergic neurons is well induced. After injection of 6-OHDA, 5 mg/kg of SMPs were intraperitoneally administered for 2 weeks. Thereafter, the test mice were fixed using 4% paraformaldehyde. Following the dissection of brain tissues from test mice, they were washed with PBS containing 4% paraformaldehyde for 24 hr and then dehydrated for 4 days using 15%, 20%, 25% and 30% sucrose solutions. The brain tissues were sliced by a freezing microtome. The brain sections (30 mm thick) were stained according to a methyl green histochemical staining method and their images were taken using a confocal microscope (LSM 510 meta, Zeiss, Feldbach, Switzerland).

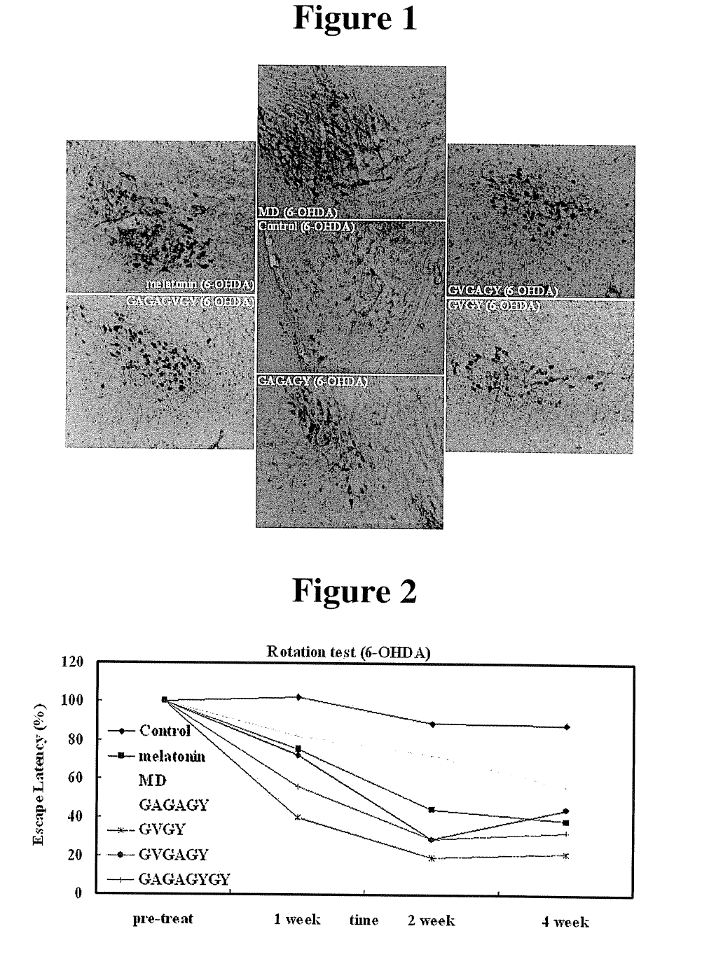

As shown in FIG. 1, dopaminergic neurons stained were not observed in brain tissues from the control (non-treatment group) injected with 6-OHDA. In contrast, the neurotoxicity of 6-OHDA was effectively prevented in the positive control MD (treated with enzymatic hydrolysate of silk protein) and the animal injected with melatonin. Likely, the mice injected with SMPs were observed to show stained dopaminergic neurons in brain tissues, demonstrating that the peptides of this invention exhibit the prevention effects on death of neuronal cells.

The MD used in the positive control denotes “BG101” that is one of enzymatic hydrolysates of silk protein described in Korean Pat. No. 0494357 filed by the present inventors.

### Example 2

### Analysis of Prevention Effect on Disorders of Behavior in Animal Models (PD Animal Models) Having Apoptotic Dopaminergic Neuronal Cells by 6-OHDA

The protection efficacy of the present peptides was tested by behavior experiments using animal models. The mice with damaged dopaminergic neurons by 6-OHDA were administered with amphetamine to induce asymmetric rotation. Then, the present peptides were examined to recover normal states.

5-week old male mice (SAMTAKO, Korea) were used. Four-week old mice were purchased and adapted for one week, followed by establishing animal models using a stereotaxic system (KOFT, CA, USA). In these experiments, 6-OHDA (3 μg/μl in normal saline) was injected into the desired region (0.0 mm posterior to bregma; 2.0 mm lateral to midline; 3.0 mm ventral to the dura) by use of the stereotaxic system, resulting in establishment of animal models. 6-OHDA was injected into substantial nigra in which neurites of dopminergic neurons are connected and sustained death of dopminergic neurons is well induced. Two weeks after 6-OHDA injection, animals were tested for amphetamine-induced turning behavior (amphetamine at 0.1 mg/kg i.p.). The mice were tested for 1.5 min. Animals with net rotational asymmetry of at least 400 full turns were selected for test. 5 mg/kg of SMPs were intraperitoneally administered for 2 weeks and their effects were examined by behavior tests. The asymmetric rotation was rapidly relieved with the lapse of time.

This experiment was performed to verify effects of silk protein-mimicking peptides in Parkinson's disease animal models. As represented in FIG. 2, all of the SMPs, MD and melatonin contribute to the relief of behavior disorders in animals by inhibiting effectively the death of dopaminergic neuronal cells.

### Example 3

### Analysis of Neuroprotective Effect on Neuronal Cell Death in Hippocampus by Amyloid Proteins

This experiment was carried out to examine whether SMPs protect neuronal toxicity of amyloid proteins in hippocampus. The amyloid proteins were injected using a stereotaxic system and death of neurons in hippocampus was visualized by methyl green.

5-week old male mice (SAMTAKO, Korea) were used. Four-week old mice were purchased and adapted for one week, followed by establishing animal disease models using a stereotaxic system (KOFT, CA, USA). In these experiments, 4 nmol/5 μl of the amyloid beta 1-42 protein (Biosource, CA, USA) was injected into a desired region by use of the stereotaxic system, resulting in establishment of animal models. The amyloid beta protein was injected into intraventricular zone in which the right and left hemispheres are connected. The right and left hemispheres were affected by the amyloid beta protein injected. Afterwards, 5 mg/kg of SMPs were intraperitoneally administered for 2 weeks. The test mice were fixed using 4% paraformaldehyde. Following the extraction of brain tissues from test mice, they were washed with PBS containing 4% paraformaldehyde for 24 hr and then dehydrated for 4 days using 15%, 20%, 25% and 30% sucrose solutions. The brain tissues were sliced by a freezing microtome. The brain sections (30 mm thick) were stained according to a methyl green histochemical staining method and their images were taken using a confocal microscope (LSM 510 meta, Zeiss, Feldbach, Switzerland).

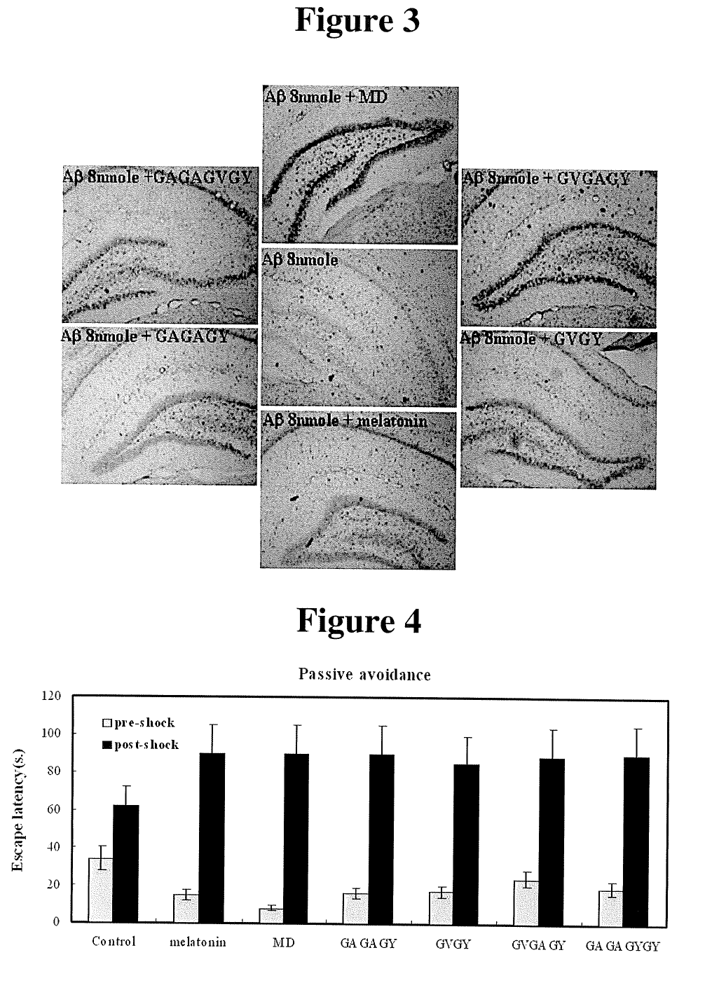

As shown in FIG. 3, the SMPs of this invention effectively prevent death of neuronal cells due to amyloid proteins. In addition, it was shown that the SMPs administered to the ventral lateral region prevent death of neurons in dentate gyrus in hippocampus.

### Example 4

### Analysis of Neuroprotective Effect on Neurotoxicity of Amyloid Proteins (Passive Avoidance Test)

This experiment was carried out according to the passive avoidance test to examine whether the SMPs prevents the declines in learning and memory abilities by neurotoxic substances. Before performing learning test, the amyloid beta protein was injected into animals through the ventral lateral region using a stereotaxic system, resulting in establishing Alzheimer's disease animal models. 3-day later, 5 mg/kg of SMPs were intraperitoneally administered for 2 weeks.

Automated shuttle box (Model PACS-30, Columbus Instruments International Company) was used as test device. The shuttle box was divided into two rooms with the same area (19″ L×9″ W×10.875″ H) by middle door (3″ L×2.625″ W), and their floors were equipped with current-generating device. Each room might be lighted a 20 W light bulb on hinged plexiglass lid. A white rat might enter a dark room through the door. Noise was control below 60 dB and the test was performed in the dark room. The rat was initially placed in a lighted room and moved to a dark room when the door was opened. At this time, the door was automatically closed and light was turned off. This test was repeated until the rat moved to the dark room within 20 seconds. 26 hours after the end of this discipline, when the rat enter the dark room, the door was closed and 1 mA of current was generated on a floor of the dark room for 3 seconds. Two weeks after injection of the amyloid beta protein through the ventral lateral region (8 nmol/ 5 μl in normal saline), the rat was placed in the lighted room, and time required for the rat to move to the dark room was measured. The time was limited to 5 minutes.

As shown in FIG. 4, the SMPs of this invention prevent the decline in learning and memory abilities by their neuroprotective activities similar to those of MD and melatonin. Under pre-shock conditions, the escape latency of rats treated with SMPs was maintained at less than 30 sec. Furthermore, their memory maintenance after 24-hr of electric shock was observed to be significant compared to the control group.

### Example 5

### Analysis of Neuroprotective Effect on Neurotoxicity of Amyloid Proteins (Water Maze Test)

This experiment was carried out according to the water maze test to examine whether the SMPs prevents the declines in learning and memory abilities by neurotoxic substances. Before performing learning test, the amyloid beta protein was injected into animals through the ventral lateral region using a stereotaxic system, resulting in establishing Alzheimer's disease animal models. 3-day later, 5 mg/kg of SMPs were intraperitoneally administered for 7 days. Firstly, the reference test was carried out for 5 days to measure learning extent and a period of time for recognizing surrounding environments and forming memory.

The rats injected with the amyloid beta protein were administered with each 5 mg/kg of SMPs for 2 weeks and then underwent the water maze test. A black-colored water pool with 120 cm-diameter was used as cages and a circular and black-colored metal platform (14×14 cm) was hidden 1 cm below the waterline. The platform was placed between walls of the water pool divided into 4 sections. The release points were randomly determined in each quadrant of the pool. The latency time was defined as the time taken to reach the platform from the release point. In addition, a swimming pattern of rats in the pool was observed using a computer equipped with an image analyzer (SMART software basic version, Frame Grabber board, Panlab s.l., Denmark). 3-sec after placing rats in the pool, all analysis and mechanical operations were performed. Where rats stay for 2 sec in the pool, record was set to automatically stop. This experiment was performed four times for four days under identical conditions with differing releasing points and finally the latency time was recorded. This analysis was considered as working memory and short-term memory. On day 5, the platform was removed and the number of times to reach a region of the platform was measured under the same conditions. This analysis was considered as reference memory and long-term memory. The longest time limit was determined 5-min. The changes in latency time indicate decline or recovery of memory ability.

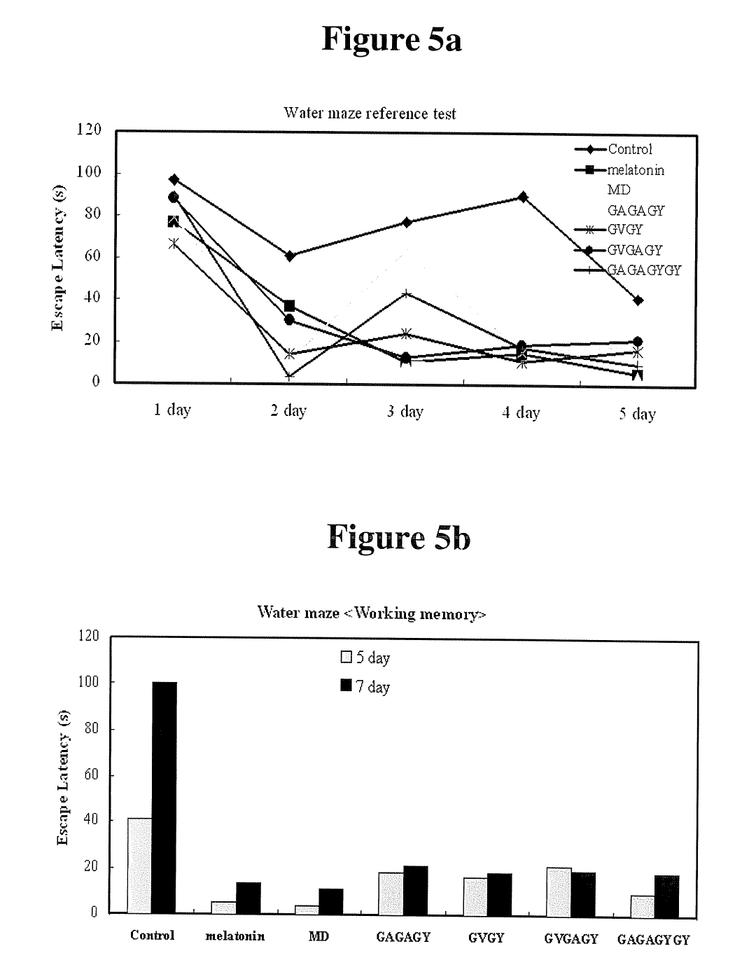

As represented in FIGS. 5a and 5b, all of the treatment groups were analyzed to show similar results. All treatment groups were determined to show substantially same results in standard deviation. Where the water maze test was re-performed at 24 hr after 5-day learning, a memory maintenance could be analyzed by working tests. As a result, it could be understood that all substances administered induced similar leaning and memory maintenance abilities.

Therefore, the results urge us to reason that the SMPs of this invention exhibit significant efficacies on learning and memory formation as well as memory maintenance by protecting neuronal cells from neurotoxins.

### Example 6

### Analysis of Neuroprotective Effect on Neuronal Cell Death by Amyloid Beta (Analysis of Neuron Viability and Nuclear Fragmentation)

The survival rate and nuclear fragmentation of neuronal cells indicating apoptosis of neuron were examined to verify the neuroprotective effects of SMPs against neurotoxic effects of the amyloid beta protein. For identifying mechanisms governing the protective effects against amyloid beta in animal tests, neurotoxic effects and nuclear damages in neuronal cell lines were examined. The substance “cosmo” used in this experiment was obtained by a preparation process identical to that for the substance MD.

The MTT reduction analysis was carried out. The human neuroblastoma cells, SK—N—SH cells (ATCC) were plated on PEI-coated 96-well plates at a density of 40,000 cells/well. The cells were cultured in DMEM supplemented with 10% FBS (fetal bovine serum). 2-hr before experiments, the medium was changed with low serum medium (DMEM with 1% FBS) and the cells were incubated with each test substance. The MTT reduction experiment was performed by slightly modifying a known method (Shearman et al., Proc. Natl. Acad. Sci. 91(4):1470-4(1994), Shearman et al., J. Neurochem. 65(1):218 27(1995), and Kaneko et al., J. Neurochem. 65(6):2585-93(1995)). Neuronal cells cultured were treated with the amyloid beta protein and incubated for 48 hr at 37° C. in 5% CO₂ incubator, followed by additional incubation for 4.5 hours with 0.5 mg/mL of MTT[3-(4,5dimeylthiazol-2-yl)-2,5 diphenyltehazolium bromide; Sigma). The treatment of SMPs was carried out for 2 hr at a 10 fM concentration before the amyloid treatment. Formazan precipitates formed by MTT reduction were dissolved in the solution (0.1 N HCl in anhydrous isopropanol), and absorbance at 570 nm was determined by ELISA

Reader. The value of the control group (only containing solvent) was determined as 100% and that of 0.9% Triton X-100-treated group as 0%. The value of each sample was relatively determined with referring the reference values.

The morphological changes of nuclear chromosomes by amyloid beta were observed by staining with DNA-binding fluorochrome bis-benz (Hoechst 33258 dye). SK—N—SH cells were incubated with 10 fM SMPs for 2 hr and then with 20 μM amyloid beta for 24 hr. 0.5-3.0×10⁶ cells treated were centrifuged at 300×g for 10 min and washed with PBS, followed by fixation with 50 μl paraformaldehyde for 10 min at room temperature. After washing with PBS, cells were incubated with 16 μg/ml bis-benzimide in PBS (15 μl) for 15 min at room temperature and their 10 μl-aliquot was placed on a slid glass, followed by the observation of apoptotic nuclear chromosome under a fluorescence microscope.

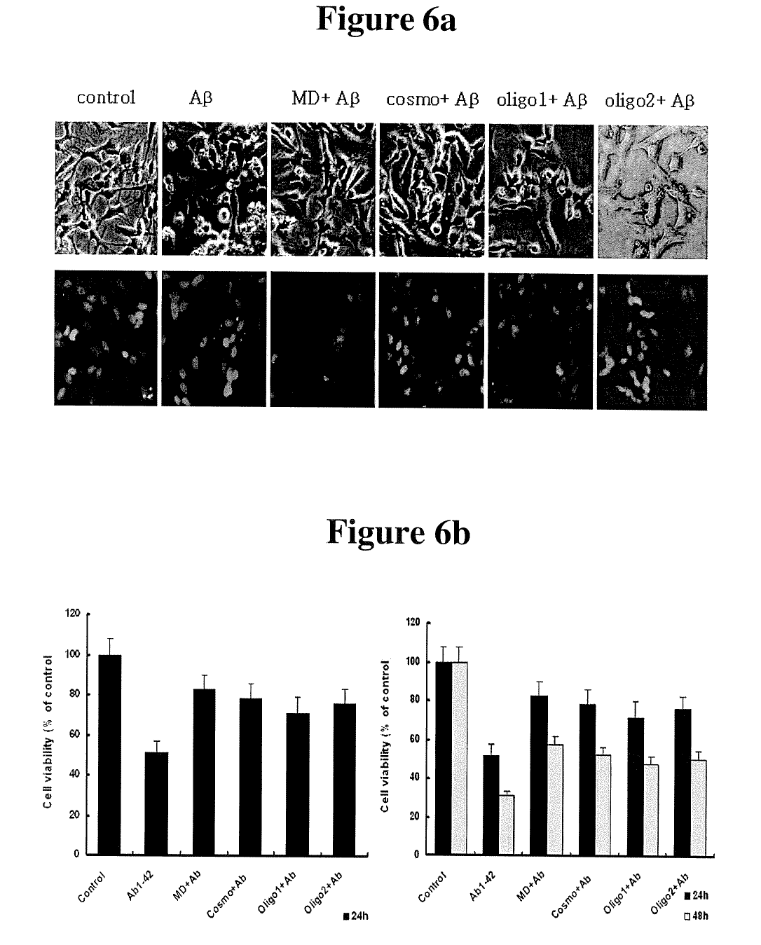

FIGS. 6a and 6b are results of nuclear fragmentation and cell viability analysis. In Figures, oliogo 1 and oligo 2 represent SMP-1 and SMP-4, respectively.

It was analyzed that the amyloid beta protein to cause Alzheimer's disease induced about 55% apoptosis in neuronal cells. The present peptides, oliogo 1 and oligo 2 dramatically inhibit apoptosis of neuronal cells similar to MD and cosmo.

In Hoechst 33258 staining experiments, SK—N—SH cells incubated with the amyloid beta protein showed serious nuclear condensation and fragmentation; however, the nuclear fragmentation was greatly decreased in cells incubated with the present peptides, oliogo 1 or oligo 2.

Consequently, it could be recognized that the SMPs of the present invention inhibit the nuclear fragmentation caused by insults to prevent apoptosis of neuronal cells, thereby exhibiting neuroprotective activities.

### Example 7

### Analysis of Neuroprotective Effect on Neuronal Cell Death by Reactive Oxygen Species

For identifying molecular mechanisms governing neuroprotective effects of the present peptides, reactive oxygen species (ROS) known to involve in neurotoxicity of amyloid beta were analyzed. Using a DCF-DA staining method, quantitative determination of reactive oxygen species was performed and microscopic observation was performed.

SK—N—SH cells (ATCC) were first treated with 10 fM SMPs for 2 hr and then with 20 μM amyloid beta protein. Afterwards, cells were incubated for 30 min at 37° C. with 10 μM DCF-DA (6-carboxy-2′,7′-dichloro-dihydrofluoresceine diacetate, dicarboxym-ethylester) dissolved in HCSS buffer solution (20 mM HEPES, 2.3 mM CaCl₂, 120 mM NaCl, 10 mM NaOH, 5 mM KC1, 1.6 mM MgCl₂, 15 mM glucose) and 2% Pluronic F-127 (suspension additive). DCF fluorescence by reactive oxygen in cells was observed at room temperature by using Olympus IX70 microscope equipped with mercury lamp fluorescence device (excitation wavelength 488 nm, emission wavelength 510 nm), and the images was taken by CCD camera and analyzed by using NIH Image 1.65 program or by flow cytometry (GENios, Tecan, NC, USA) at excitation wavelength of 485 nm and emission wavelength of 510 nm.

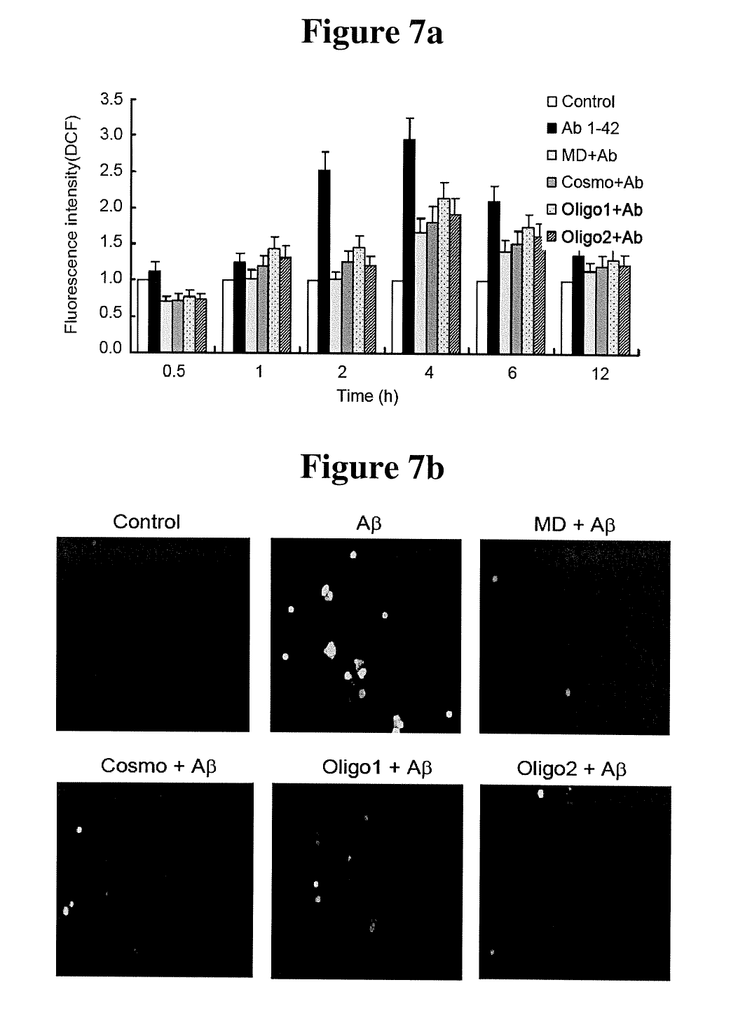

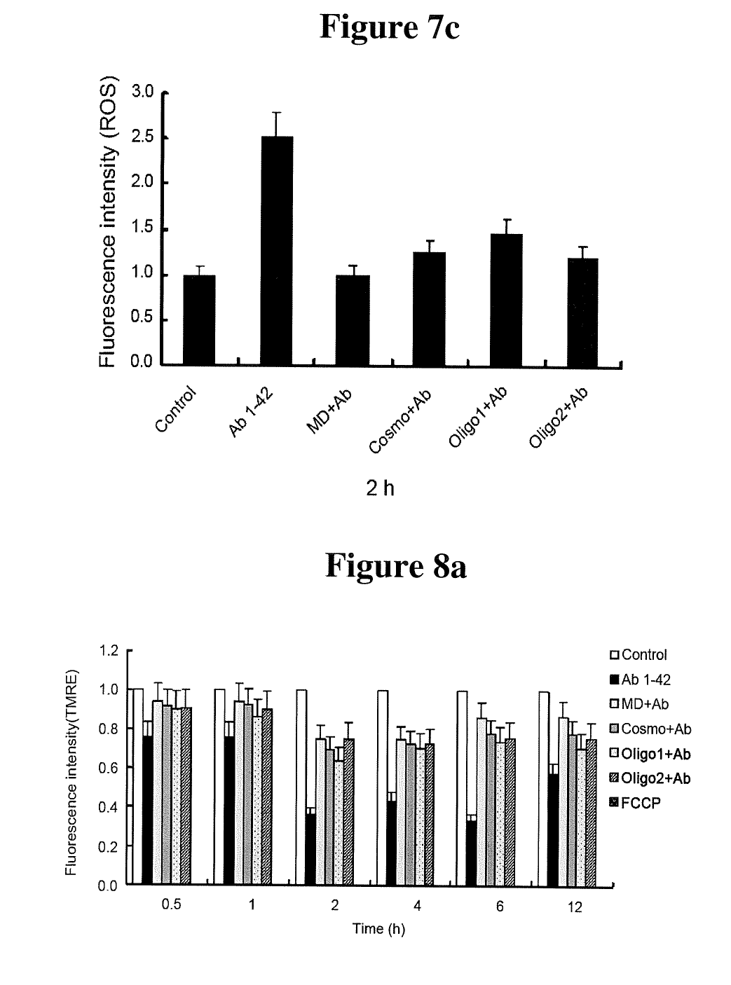

As shown in FIG. 7a, all of the present peptides (oliogo 1 and oligo 2), MD and cosmo result in the decrease in levels of reactive oxygen species at similar extent. The oligo 2 shows a little enhanced effects compared to the oligo 1. These results are consistent with those of animal behavior tests. In the fluorescence microscopic analysis, it was clearly observed that the present peptides effectively inhibited levels of reactive oxygen species.

Consequently, it could be appreciated that the peptides of this invention inhibits the generation of reactive oxygen species to prevent apoptosis of neuronal cells.

### Example 8

### Analysis of Neuroprotective Effect on Neuronal Cell Death by Impairment of Mitochondrial Functions

We examined the influence of the present peptides on subcellular mitochondria to play a crucial role in cellular energy generation. It have been already reported that the amyloid beta protein induces elevation of reactive oxygen species in cells and simultaneously results in nuclear fragmentation, dysfunction of mitochondrial membrane and loss of mitochondrial functions. In this regard, we examined the influence of the present peptides on mitochondrial functions relating closely to cell viability.

Using a fluorescent staining with TMRE (tetramethyl rhodamine-ethylester, Molecular Probe), the disruption of mitochondrial membrane potential was measured. The positively charged TMRE is penetrated into mitochondrial membrane with help of mitochondrial membrane potential, and the fluorescent intensities indicate the maintenance of mitochondrial membrane potential. SK—N—SH cells (ATCC) were first treated with 10 fM SMPs for 2 hr and then with 20 μM amyloid beta protein for 6 hr. Afterwards, cells were incubated for 15 min at 37° C. with 100 nM TMRE and the fluorescent intensities were measured using a fluoremetry at excitation wavelength of 549 nm and emission wavelength of 574 nm. The fluorescent images were taken using a fluorescence microscope (Olympus IX70) and CCD camera.

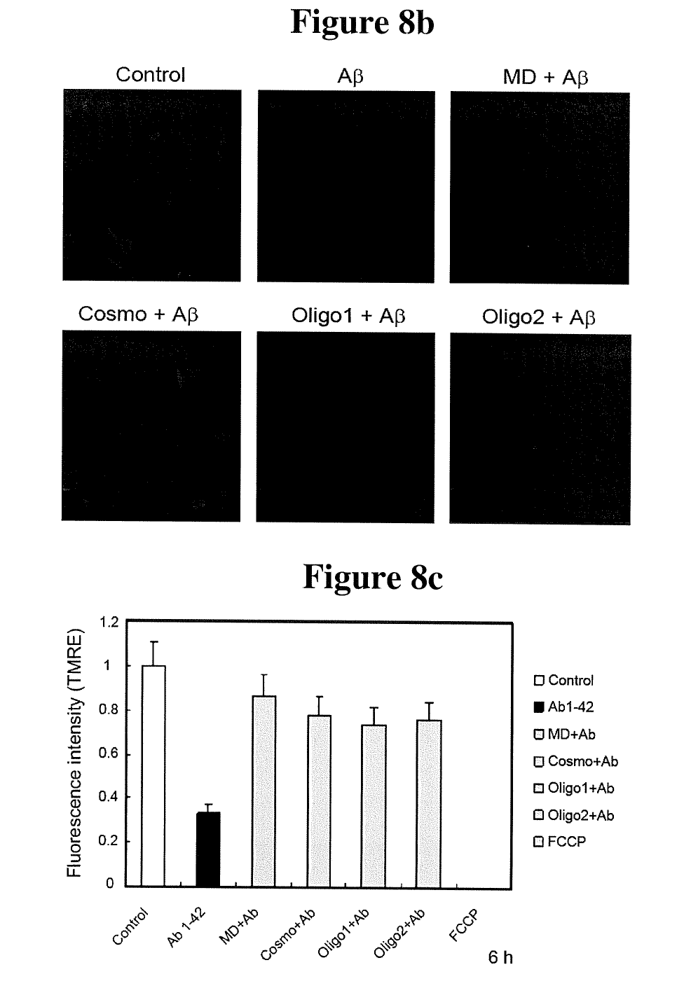

It was shown that the treatment of amyloid beta gave rise to the decrease in TMRE fluorescence intensity to indicate collapse of mitochondrial membrane potential (FIG. 8a). In contrast, cells incubated with the present peptides (oligo 1 and oligo 2) showed little or no abnormality of mitochondrial membrane, which was confirmed by fluorescence microscopic observation (FIGS. 8a-8c).

These results demonstrate that the present peptides (oligo 1 and oligo 2) protect mitochondrial functions to play a crucial role in cell viability. In other words, it could be appreciated that the present peptides are responsible for maintenance of mitochondrial functions to protect cells from neurotoxicity such as amyloid beta, thereby finally inhibiting apoptosis of neuronal cells.

### Example 9

### Analysis of Neuroprotective Effect on Neuronal Damages Caused by High Glucose Level

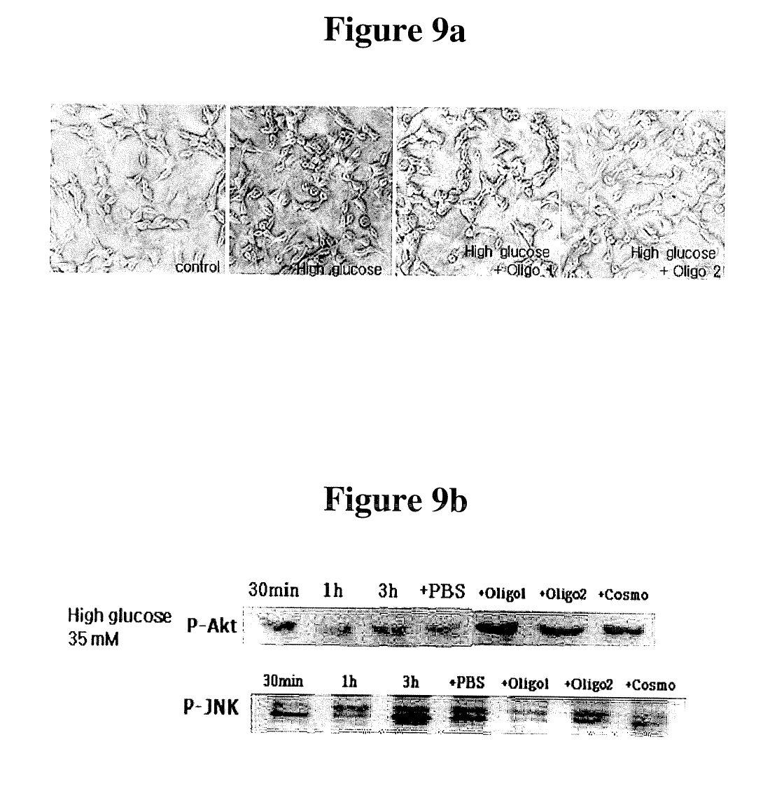

Diabetes characterized by high level of glucose causes events to damage cells and tissues. This experiment is to analyze neuroprotective activities of SMPs on SK—N—SH neuronal cells in high level of glucose (35 mM). As shown in FIG. 9a, the treatment of high glucose level causes apoptosis of neuronal cells after 3 hr. In contrast, the treatment of the present peptides, oligo 1 and oligo 2 effectively inhibits apoptosis. The cell viability was shown to be over 30%.

To identify molecular mechanisms governing neuroprotective activities of the present peptides on apoptosis of neuronal cells caused by high glucose level, p-Akt and p-JNK were analyzed on electrophoresis.

As shown in FIG. 9b, 35 mM glucose continuously decreases the level of p-Akt from post-30 min treatment. At 3-hr treatment, the level of p-Akt was shown to be the lowest. However, the treatment of the present peptides dramatically enhances the level of p-Akt to play an important role in cell viability. In particular, the oligo 1 gives rise to the highest level of p-Akt. Other peptides enhance the expression of p-Akt at similar extent. The expression of p-JNK to play a crucial role in apoptosis was also analyzed on electrophoresis. As represented in FIG. 9b, at 3-hr treatment of high glucose level, the level of p-JNK was shown to be the highest. In contrast, the treatment of the present peptides effectively inhibits the level of p-JNK.

Consequently, it could be understood that the present peptides contribute to the increase in the level of p-Akt and the decrease in the level of p-JNK, resulting in effective prevention of apoptosis of neuronal cells. In addition, these experimental results demonstrate that the present peptides could prevent damages of neuronal cells of diabetic patients exposed high glucose level and effectively inhibit apoptosis of neuronal cells.

### Example 10

### Analysis of Neuroprotective Effect on Traumatic Neuronal Damages Imposed Directly on Brain

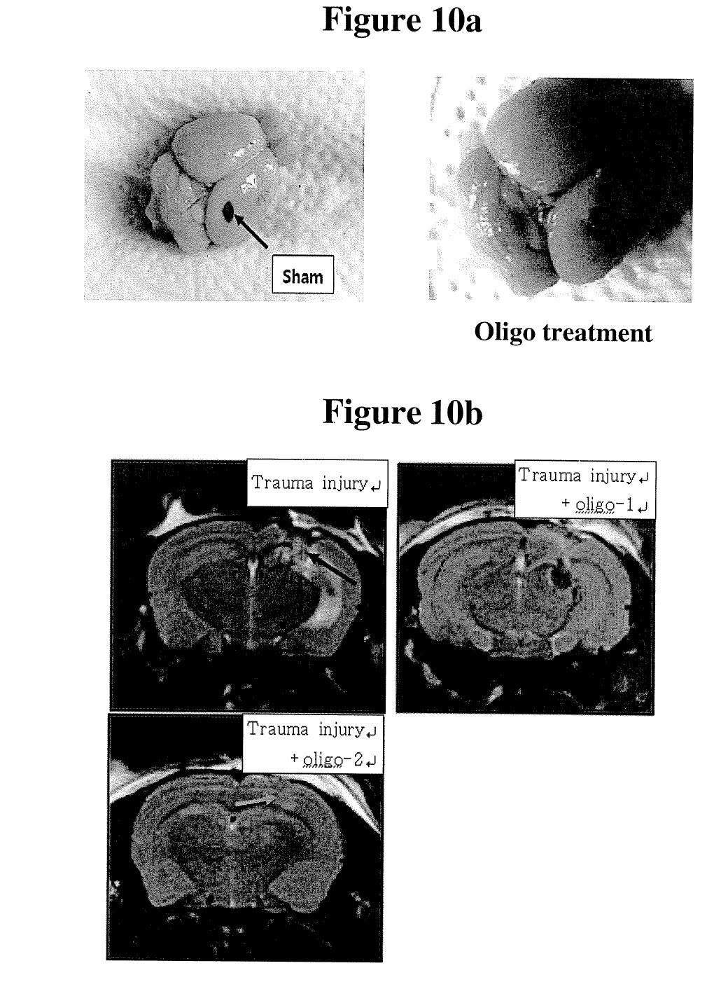

To examine whether physical shocked-brain could be protected by the present peptides, the test was performed using five-week old mice. Totally, 17 gauge needle tracks were formed to induce trauma and then the present peptides were examined to prevent trauma and inflammation. Each experimental group consists of five mice and the results are statistically calculated using T-test. The results were obtained after 4-week physical shock. In the event that brain was physically shocked and then PBS was injected into a damaged region (Sham group), the damage became worse due to inflammation. However, the treatment of the present peptides effectively ameliorates the damage (FIG. 10a). Surprisingly, the amelioration of traumatic damages was clearly observed. To accurately analyze traumatic regions, 4.7T MRI (magnetic resonance imaging) was carried out (FIG. 10b). As represented in FIG. 10b, the physical shocked-region became necrotic; however, the injection of the present peptides after traumatic damage effectively prevents such damages.

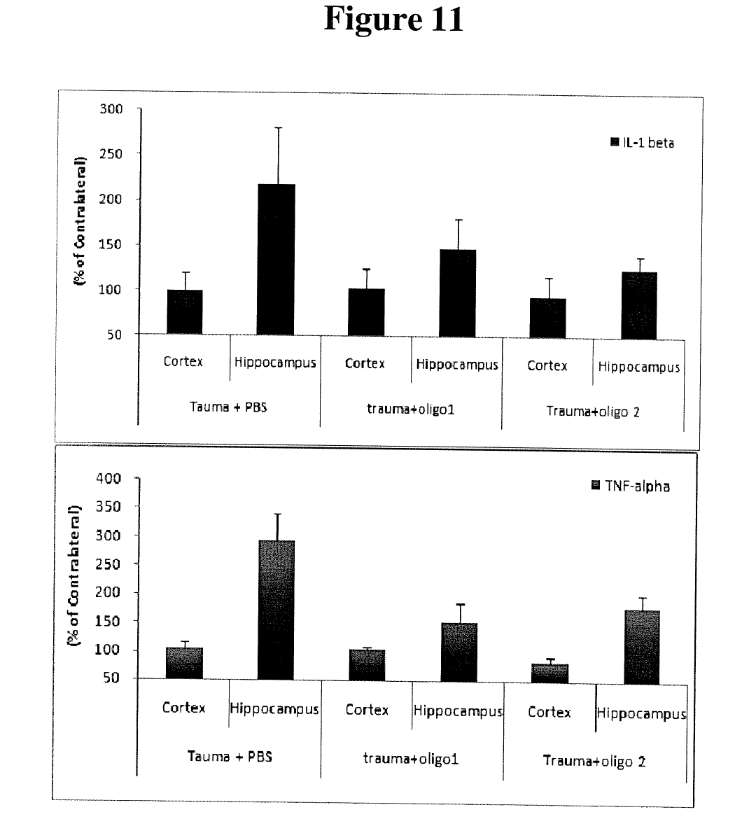

We further identified molecules involved in the treatment of traumatic damages. On the basis of the experimental results that the present peptides inhibits apoptosis of neuronal cells and influence of neurotoxicities such as reactive oxygen species, the influence of the present peptides on pro-inflammatory cytokines such as interleukin-1β and TNF-α was analyzed using ELISA-detection kits. The brain was imparted by traumatic damages and then 12-hr later, brain tissues were taken. The cortex of the damaged region was shown to be tremendous damages such that cytokine analysis of this region was not able to perform. In the hippocampus showing active inflammation owing to traumatic damage, pro-inflammatory cytokines were detected in high levels. However, the treatment of the present peptides effectively inhibits level of IL-1β (FIG. 11). The similar profile to IL-1β was measured for TNF-α (FIG. 11). The difference in the profile of TNF-α was also observed more clearly in the hippocampus than the cortex having tremendous damages. The present peptides were determined to inhibit level of the pro-inflammatory cytokine TNF-α in regions surrounding the damaged hippocampus.

Interestingly, these results address that the present peptides inhibits inflammation induced by traumatic damages. In particular, they could prevent the induction of damages of surrounding regions around initially damaged region to effectively treat damaged regions.

Having described a preferred embodiment of the present invention, it is to be understood that variants and modifications thereof falling within the spirit of the invention may become apparent to those skilled in this art, and the scope of this invention is to be determined by appended claims and their equivalents.

1. A silk protein-mimicking peptide represented by the following Formula 1:
Gly-Xₐₐ₁-Gly-Xₐₐ₂ (1)
wherein Xₐₐ₁ is Ala, Val, Ser, Tyr, Asp, Glu, Thr, Met, Ile, Leu, Phe, His, Lys or Arg; Xₐₐ₂ is Ala, Tyr, Val, Ser, Asp, Glu, Thr, Met, Ile, Leu, Phe, His, Lys or Arg; or Xₐₐ₂ represents Ala, Tyr, Val, Ser, Asp, Glu, Thr, Met, Ile, Leu, Phe, His, Lys or Arg residue linked to Gly-Xₐₐ₃; and Xₐₐ₃ is Tyr, Val, Ala, Ser, Asp, Glu, Thr, Met, Ile, Leu, Phe, His, Lys or Arg.

2. The peptide according to claim 1, wherein Xₐₐ₁ is Ala, Val, Ser or Tyr.

3. The peptide according to claim 1, wherein Xₐₐ₂ is Ala, Tyr, Val or Ser.

4. The peptide according to claim 1, wherein Xₐₐ₂ is Ala, Tyr, Val or Ser residue linked to Gly-Xₐₐ₃; and Xₐₐ₃ is Tyr, Val, Ala or Ser.

5. The peptide according to claim 4, wherein Xₐₐ₃ further comprises Gly-Xₐₐ₄; and Xₐₐ₄ is Tyr, Ala, Val, Ser, Asp, Glu, Thr, Met, Ile, Leu, Phe, His, Lys or Arg.

6. The peptide according to claim 1, wherein the peptide comprises the amino acid sequence selected from the group consisting of SEQ ID NOs. 1-4.

7. A composition for preventing or treating a brain disease, comprising an effective amount of the silk protein-mimicking peptide of claim 1.

8. The composition according to claim 7, wherein the composition is a pharmaceutical composition or a food composition.

9. The composition according to claim 7, wherein the brain disease is selected from the group consisting of neurodegenerative diseases, ischemia-reperfusion injury and mental disorders.

10. The composition according to claim 9, wherein the neurodegenerative disease is dementia, Huntington's disease, Parkinson's disease or amyotrophic lateral sclerosis.

11. The composition according to claim 9, wherein the ischemia-reperfusion injury is ischemic stroke.

12. The composition according to claim 9, wherein the mental disorder is depression, schizophrenia and post traumatic stress disorder.

13. The composition according to claim 7, wherein the peptide has a neuroprotective activity.

14. The composition according to claim 13, wherein the peptide inhibits apoptosis of neuronal cells to exhibit neuroprotective activity.

15. The composition according to claim 14, wherein the peptide inhibits the generation of reactive oxygen species or protects mitochondrial functions to exhibit neuroprotective activity.

16. A composition for improving a brain or cognitive function, comprising an effective amount of the silk protein-mimicking peptide of claim 1.

17. The composition according to claim 16, wherein the composition is a pharmaceutical composition or a food composition.

18. The composition according to claim 16, wherein the brain or cognitive function is a learning ability, a memory ability or a concentration ability.

19. The composition according to claim 16, wherein the composition ameliorates the aggravation of brain or cognitive functions associated with brain diseases.

20. The composition according to claim 16, wherein the peptide has a neuroprotective activity.

21. The composition according to claim 20, wherein the peptide inhibits apoptosis of neuronal cells to exhibit neuroprotective activity.

22. The composition according to claim 21, wherein the peptide inhibits the generation of reactive oxygen species or protects mitochondrial functions to exhibit neuroprotective activity.

23. A composition for preventing or treating diseases, disorders or conditions associated with oxidative stress, comprising an effective amount of the silk protein-mimicking peptide of claim 1.

24. The composition according to claim 23, wherein the composition is a pharmaceutical composition or a food composition.

25. The composition according to claim 23, wherein the disease, disorder or condition associated with oxidative stress is aging; central nerve system disorders including trauma, cerebral palsy and diabetic neuropathy; cardiovascular diseases including intermittent claudication and arteriosclerosis; cataract; musculoskeletal diseases including arthritis; or disorders associated with environments causing the generation of reactive oxygen species including ionizing radiation-associated disorder, cancer chemotherapy-associated disorder and carcinogen expose-associated disorder.

26. The composition according to claim 23, wherein the peptide inhibits the generation of interleukin-1β (IL-1β) or tumor necrosis factor-α (TNF-α).

27. A method for preventing or treating a brain disease, comprising administering to a subject a composition comprising an effective amount of the silk protein-mimicking peptide of claim 1.

28. The method according to claim 27, wherein the composition is a pharmaceutical composition or a food composition.

29. The method according to claim 27, wherein the brain disease is selected from the group consisting of neurodegenerative diseases, ischemia-reperfusion injury and mental disorders.

30. The method according to claim 29, wherein the neurodegenerative disease is dementia, Huntington's disease, Parkinson's disease or amyotrophic lateral sclerosis.

31. The method according to claim 29, wherein the ischemia-reperfusion injury is ischemic stroke.

32. The method according to claim 29, wherein the mental disorder is depression, schizophrenia and post traumatic stress disorder.

33. The method according to claim 27, wherein the peptide has a neuroprotective activity.

34. The method according to claim 33, wherein the peptide inhibits apoptosis of neuronal cells to exhibit neuroprotective activity.

35. The method according to claim 34, wherein the peptide inhibits the generation of reactive oxygen species or protects mitochondrial functions to exhibit neuroprotective activity.

36. A method for improving a brain or cognitive function, comprising administering to a subject a composition comprising an effective amount of the silk protein-mimicking peptide of claim 1.

37. The method according to claim 36, wherein the composition is a pharmaceutical composition or a food composition.

38. The method according to claim 36, wherein the brain or cognitive function is a learning ability, a memory ability or a concentration ability.

39. The method according to claim 36, wherein the composition ameliorates the aggravation of brain or cognitive functions associated with brain diseases.

40. The method according to claim 36, wherein the peptide has a neuroprotective activity.

41. The method according to claim 40, wherein the peptide inhibits apoptosis of neuronal cells to exhibit neuroprotective activity.

42. The method according to claim 41, wherein the peptide inhibits the generation of reactive oxygen species or protects mitochondrial functions to exhibit neuroprotective activity.

43. A method for preventing or treating diseases, disorders or conditions associated with oxidative stress, comprising administering to a subject a composition comprising an effective amount of the silk protein-mimicking peptide of claim 1.

44. The method according to claim 43, wherein the composition is a pharmaceutical composition or a food composition.

45. The method according to claim 43, wherein the disease, disorder or condition associated with oxidative stress is aging; central nerve system disorders including trauma, cerebral palsy and diabetic neuropathy; cardiovascular diseases including intermittent claudication and arteriosclerosis; cataract; musculoskeletal diseases including arthritis; or disorders associated with environments causing the generation of reactive oxygen species including ionizing radiation-associated disorder, cancer chemotherapy-associated disorder and carcinogen expose-associated disorder.

46. The method according to claim 43, wherein the peptide inhibits the generation of interleukin-1β (IL-1β) or tumor necrosis factor-α (TNF-α).


SEQUENCE LISTING (4 sequences)

SEQ ID NO: 1 - protein, 8 aa, Artificial Sequence
  SMP-1
GAGAGVGY

SEQ ID NO: 2 - protein, 4 aa, Artificial Sequence
  SMP-2
GVGY

SEQ ID NO: 3 - protein, 6 aa, Artificial Sequence
  SMP-3
GAGAGY

SEQ ID NO: 4 - protein, 6 aa, Artificial Sequence
  SMP-4
GVGAGY